(19) United States
(12) Patent Application Publication
(10) Pub. No.: US 2007/0184124 A1
(43) Pub. Date: Aug. 9, 2007

(54) Skin stabilizer preparation

(76) Inventor: Steve Harlan, Des Moines, IA (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/348,503

(22) Filed: Feb. 7, 2006

(51) Int. Cl. A61K 33/04; A61K 31/573

(52) U.S. Cl. 424/703; 514/171

(57) ABSTRACT

A skin stabilizer system is provided which comprises separate compositions of a corticosteroid and sulfur. The compositions may be combined in any desired ratio by the user, and applied to the skin.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention generally relates to a skin stabilizer system to aid in the treatment and prevention of various skin conditions. More particularly, the present invention relates to a novel skin stabilizer system containing a corticosteroid and sulfur.

2. Description of the Related Art

Many people suffer from various kinds of adverse skin conditions, such as itching, “combination skin,” red bumpy skin around the nose, mouth and eyelids (perioral dermatitis), mild acne, irritation from acne medication, shaving rashes on the face and neck, underarms, legs and bikini line, redness from rosacea, redness from sun damage, and rough painful spots from sun damage (actinic keratoses), psoriasis, and eczema. Although there are many prescription medications on the market to treat these kinds of unfavorable symptoms and skin conditions, many of these medications are not recommended or allowed for use over an extended period of time despite the fact that those adverse symptoms are chronic and recurrent or take a very long time to heal or control. Therefore, there is a need for a skin system that can be used for an extended period of time, including indefinitely, to control unfavorable skin symptoms.

Further, some of adverse skin conditions are side effects of strong topical medications used to treat other adverse skin conditions, for example, irritation from topical 5-FU treatment of actinic keratoses on the face and arms, and irritation from retinoids. Once such an adverse symptom from a strong skin medication occurs or a patient finishes his or her prescription of a strong medication, unfortunately the patient's only available option is to stop ‘cold turkey.’ Therefore, there is a need for a skin stabilizer system for transitioning from a strong prescription medication for an immediate improvement of skin condition to a mild skin preparation to maintain and stabilize skin condition.

Often patients with very sensitive skin cannot tolerate commonly used topical medications such as Benzamycin gel (trademark), Spear Tretinoin (trademark), RetinA (trademark), and benzoyl peroxide products, metronidazole preparations, and alpha hydroxyl acid preparations. Therefore, there is a need for a system that stabilizes the skin for other skin care medications.

### BRIEF SUMMARY OF THE INVENTION

In one aspect, the present invention provides a system for the treatment of a skin condition by a user in need of such treatment, said system comprising:
a. a composition which comprises a corticosteroid as the active ingredient; and
b. a composition which comprises sulfur as the active ingredient; wherein said compositions are physically separated prior to use by the user.

In another aspect, the present invention provides a method of preparing a system for the treatment of a skin condition by a user in need of such treatment, said method comprising:
a. providing a composition which comprises a corticosteroid as the active ingredient;
b. providing a composition which comprises sulfur as the active ingredient; and
c. associating said compositions such that said compositions are accessible to the user but are physically separated prior to use by the user.

In yet another aspect, the present invention provides a method for the treatment of a skin condition in a user in need of such treatment, said method comprising:
a. providing the system described above;
b. determining the relative amounts of each composition to be applied to the skin; and
c. applying the determined relative amounts of each composition to the skin.

In yet another aspect, the present invention provides a method for determining a suitable composition for application to the skin of a user in need of such application, said method comprising:
a. providing the system described above;
b. forming a first composition by mixing an amount of the corticosteroid composition and an amount of the sulfur composition, wherein the first composition formed has a higher proportion of corticosteroid and a lower proportion of sulfur as compared to subsequently formed compositions;
c. applying said first composition to the skin of said user;
d. determining the user's tolerance and response for said first composition;
e. if the first composition is tolerated by the user, forming a second composition by mixing an amount of the corticosteroid composition and an amount of the sulfur composition, wherein the second composition has a lower proportion of corticosteroid and a higher proportion of sulfur as compared to the first composition.

### DETAILED DESCRIPTION OF THE INVENTION

An important aspect of the systems of the present invention is that they contain separate compositions of a corticosteroid and sulfur. Thus, the system may comprise the compositions contained in separate containers. More preferably, they are contained in separate containers bound together, e.g., two pump bottles packaged together. Most preferably, they are contained in two separate compartments within a container with a pump for each compartment. In a preferred embodiment, the system also contains instructional material for use of the system. The particular packaging means is not critical, as long as the individual compositions are kept separate until use. Further, the size of the system is not critical. In a preferred embodiment the system comprises two containers holding about two ounces to about four ounces of each composition.

Each separate container or compartment of the system may have its own content-dispensing part such as pump to facilitate accurate measurement. Therefore, when a user mixes the corticosteroid composition and the sulfur composition in a certain ratio, the user can simplify the measurement by relying on how many times he or she presses each pump. For example, for a one-to-two ratio, one can simply press one pump once and then press the other pump twice.

The separate corticosteroid composition is preferably prescription strength, although a non-prescription strength corticosteroid composition is still contemplated in the invention. The corticosteroid is preferably present in the composition in an amount of from about 0.75% to about 2.0% (weight/weight (“w/w”)), more preferably about 1.5% (w/w).

The sulfur in the separate sulfur composition is preferably present in an amount of from about 0.25% to about 2.0%, more preferably about 0.75%.

Many corticosteroids are suitable for use in connection with the present invention, including, for example, hydrocortisone, hydrocortisone acetate, 0.05% triamcinolone, 0.075% betamethasone dipropionate, and 0.04% clobetasol. The sulfur component may be any suitable form, including precipitated sulfur.

In use, a small portion of each of the separate corticosteroid and sulfur compositions is mixed as needed, preferably right before application. The exact ratio of the two compositions may be directed by the user's pharmacist or physician, and can be varied as the user's skin condition changes. For example, in a preferred embodiment, the user starts out with a mixture containing a relatively high amount of the corticosteroid and a relatively low amount of the sulfur composition. That mixture is used for a period of time and the effect on the user's skin is evaluated. If that particular mixture is well-tolerated, the user may adjust the composition of the mixture to increase the amount of sulfur and to decrease the amount of corticosteroid. That process may be repeated numerous times until the user is able to use a composition that has a relatively low proportion of the corticosteroid and a relatively high proportion of the sulfur lotion. It is contemplated that the user would apply the mixture to necessary areas of the skin at least once daily, preferably two times daily. It is also contemplated that each particular mixture will be evaluated for a period of at least two weeks, preferably 2 to 4 weeks, although longer or shorter periods are possible depending on the user's skin condition and response to the particular mixture being used.

The system of the present invention may be used in the treatment of a wide variety of skin conditions, including itching, perioral dermatitis, psoriasis, eczema, pruritis, seborrheic dermatitis, acne, weed rashes, skin irritation, skin scaling, and sun damage.

The various ratios of corticosteroid and sulfur can be tried to optimize the ratio of the two ingredients for individual differences and even different stages of an individual's skin improvement. Generally, one part of corticosteroid composition is mixed with one part of the sulfur composition for most skin conditions including seborrheic dermatitis and perioral dermatitis (also known as combination skin), one part of corticosteroid composition is mixed with two parts of the sulfur composition for oily skin or rosacea, and two parts of corticosteroid composition are mixed with one part of the sulfur composition for extremely sensitive skin and eczema. A diverse ratio from 1:20 to 20:1, however, can be used for optimal results, based on the individual user's condition and response. The present invention is preferably used to treat facial skin, although it is applicable to other skin areas as well.

The following is a non-limiting example of a hydrocortisone composition for the skin stabilizer preparation.

| | |
|---|---|
| Ingredient | Unit (w/w) |

Phase 1

| | |
|---|---|
| Water | Qs 100% w/w |
| Potassium sorbate | 0.2 |
| Carbomer 934 | 0.3 |

Phase 2

| | |
|---|---|
| Glycerin | 3.0 |
| Magnesium Aluminum Silicate | 1.0 |

Phase 3

| | |
|---|---|
| Petrolatum | 6.0 |
| Dimethicone | 3.0 |
| Steareth-2 | 3.0 |
| Laureth-23 | 1.0 |
| Cetyl alcohol | 1.0 |

Phase 4

| | |
|---|---|
| Water | 2.0 |
| Glycerin | 2.0 |
| Hydrocortisone USP | 0.75 |

Phase 5

| | |
|---|---|
| Water | 1.0 |
| Benzyl alcohol | 1.0 |

Phase 6

| | |
|---|---|
| Sodium hydroxide, 50% sol | qs pH ˜6.5-7.0 |

The following is a non-limiting example of the compounding procedure to prepare a hydrocortisone composition for the skin stabilizer preparation.
1. Disperse Carbomer in Phase 1 water.
2. Disperse Mg Al Silicate in Phase 2 Glycerin.
3. Add Phase 2 to Phase 1 and heat with non-vortex mixing to 60-90° C., preferably 70° C.
4. Combine Phase 3 ingredients and heat to 60-90° C., preferably 72° C.
5. Add Phase 3 to Phase 1+2 to form emulsion.
6. Cool emulsion to 35-55° C., preferably 40° C.
7. Combine Phase 4 ingredients to form a slurry, and add to emulsion.
8. Homogenize emulsion to completely disperse actives.
9. Combine Phase 5 ingredients and add to emulsion.
10. Adjust emulsion pH to 6.5-7, preferably 7 using NaOH 50% aqueous solution.
11. As emulsion thickens, change from propellor to sidesweep mixing and continue cooling to ambient temperature. Add water to reach a specific gravity of about 0.95.

The following is a non-limiting example of the elemental sulfur composition for the skin stabilizer preparation.

| | |
|---|---|
| Ingredient | Unit (w/w) |

Phase 1

| | |
|---|---|
| Water | qs 100% w/w |
| Potassium sorbate | 0.2 |
| Carbomer 934 | 0.3 |

Phase 2

| | |
|---|---|
| Glycerin | 3.0 |
| Magnesium Aluminum Silicate | 1.0 |

Phase 3

| | |
|---|---|
| Petrolatum | 6.0 |
| Dimethicone | 3.0 |
| Steareth-2 | 3.0 |
| Laureth-23 | 1.0 |
| Cetyl alcohol | 1.0 |

Phase 4

| | |
|---|---|
| Water | 2.0 |
| Glycerin | 2.0 |
| Precipitated sulfur | 0.75 |

Phase 5

| | |
|---|---|
| Water | 1.0 |
| Benzyl alcohol | 1.0 |

Phase 6

| | |
|---|---|
| Sodium hydroxide, 50% sol | Qs pH ˜6.5-7 |

The following is a non-limiting example of the compounding procedure to prepare an elemental sulfur composition for the skin stabilizer preparation.
1. Disperse Carbomer in Phase 1 water.
2. Disperse Mg Al Silicate in Phase 2 Glycerin.
3. Add Phase 2 to Phase 1 and heat with non-vortex mixing to 60-90° C., preferably 70° C.
4. Combine Phase 3 ingredients and heat to 60-90° C., preferably 72° C.
5. Add Phase 3 to Phase 1+2 to form emulsion.
6. Cool emulsion to 35-55° C., preferably 40° C.
7. Combine Phase 4 ingredients to form a slurry. Add to emulsion.
8. Homogenize emulsion to completely disperse actives.
9. Combine Phase 5 ingredients and add to emulsion.
10. Adjust emulsion pH to 6.5-7, preferably to 7 using NaOH 50% aqueous solution.
11. As emulsion thickens, change from propellor to sidesweep mixing and continue cooling to ambient temperature. Add water to reach a specific gravity of about 0.95.

The skin stabilizer preparation can also be used as a transition product of calcineurin inhibitors for eczema and contact dermatitis such as Elidel (trademark) and Protopic™.

The stabilizer prevents and treats irritation from strong prescription topical medications. Patients who are unable to tolerate Benzamycin gel (trademark) therapy for acne therapy can continue on their prescriptions of Benzamycin gel when they use a small amount of the skin stabilizer preparation once or twice daily. This also true for patients with sensitive skin and/or “combination skin” who have been prescribed Spear Tretinoin (trademark), RetinA (trademark), and benzoyl peroxide products including the Benza-products for acne, the Metronidazole products for rosacea, and topical 5-FU products for actinic keratoses.

The inclusion of elemental sulfur (PS) in the skin stabilizer preparation inhibits the overgrowth of Pityrosporum yeast and bacteria in the skin. This allows relatively lower dosages of corticosteroid in the stabilizer system to be used safely for an extended period of time (almost indefinetly). In a clinical review of a thousand patients with long term use, there were no incidents of steroid dermatitis or steroid side effect. Therefore, people with some chronic skin conditions or a skin condition that takes a very long time to treat or stabilize, for example, eczema, psoriasis, people with “combination skin,” red bumpy skin around the nose and mouth, mild acne, shaving rashes, redness from rosacea, and sun damaged red rough skin, will consistently benefit from the skin stabilizer. The stabilizer does not contain sulfacetamide and can be used by patients allergic to the sulfa anti-biotics, but can contain safe natural ingredients including fragrance. Further, a preferred embodiment of the current invention does not contain a medication which often causes skin irritation (e.g. salicylic acid) to maximize the stabilization effect of the preparation.

Because the skin stabilizer preparation contains a lower and safer dose of corticosteroid and precipitated sulfur, it enables users to minimize adverse side effects from which users often suffer when they are exposed to a strong dose of corticosteroid or sulfuir over an extended period of time. The stabilizer preparation of the invention is safer in terms of adverse effects, but often is just as effective as high dose or full dose of corticosteroid or sulfur for many users. The low doses of corticosteroid and sulfur produce efficacy, prevents development of resistance, and provides more safety from side effects.

While preferred embodiments of the present invention have been described above, it should be understood that they have been presented by way of example only, and not limitation. Thus, the breadth and scope of the present invention should not be limited by the above described exemplary embodiments.

Obviously, numerous modifications and variations of the present invention are possible in light of the above teachings. It is therefore to be understood that the invention may be practiced otherwise than as specifically described herein.

1. A system for the treatment of a skin condition by a user in need of such treatment, said system comprising:
a. a composition which comprises a corticosteroid as the active ingredient; and
b. a composition which comprises sulfur as the active ingredient; wherein said compositions are physically separated prior to use by the user.

2. The system of claim 1, wherein each composition is contained in separate containers associated with each other, and wherein each of said containers comprises means for dispensing the composition contained therein.

3. The system of claim 2, wherein each composition is contained in a separate pump bottle, and said pump bottles are joined in a box.

4. The system of claim 1, wherein the compositions are contained in separate compartments within a vessel.

5. The system of claim 1, wherein the corticosteroid comprises hydrocortisone, triamcinolone, betamethasone dipropionate, or clobetasol.

6. The system of claim 1, wherein the corticosteroid comprises hydrocortisone acetate.

7. The system of claim 1, wherein the sulfur comprises precipitated sulfur.

8. The system of claim 1, wherein the concentration of the corticosteroid in the composition is from about 0.75% to about 2.0%.

9. The system of claim 8, wherein the concentration of the corticosteroid in the composition is about 1.5%.

10. The system of claim 1, wherein the concentration of the sulfur in the composition is from about 0.25% to about 2.0%.

11. The system of claim 10, wherein the concentration of the sulfur in the composition is about 0.75%.

12. The system of claim 1, which further comprises instructional material for the use of said system.

13. The system of claim 1, wherein the condition is selected from the group consisting of itching, perioral dermatitis, eczema, psorasis, pruritis, seborrheic dermatitis, acne, rashes, skin irritation, skin scaling and sun damage.

14. The system of claim 1, wherein the user is able to vary the relative amount of each composition to be applied to the skin.

15. A method of preparing a system for the treatment of a skin condition by a user in need of such treatment, said method comprising:
a. providing a composition which comprises a corticosteroid as the active ingredient;
b. providing a composition which comprises sulfur as the active ingredient; and
c. associating said compositions such that said compositions are accessible to the user but are physically separated prior to use by the user.

16. The method of claim 15, wherein each composition is contained in separate containers associated with each other, and wherein each of said containers comprises means for dispensing the composition contained therein.

17. The method of claim 15, wherein each composition is contained in a separate pump bottle, and said pump bottles are packaged together in a box, or a tray.

18. The method of claim 15, wherein the compositions are contained in separate compartments within a vessel.

19. The method of claim 15, wherein the corticosteroid comprises hydrocortisone, triamcinolone, betamethasone dipropionate, or clobetasol.

20. The method of claim 15, wherein the corticosteroid comprises hydrocortisone acetate.

21. The method of claim 15, wherein the sulfur comprises precipitated sulfur.

22. The method of claim 15, wherein the concentration of the corticosteroid in the composition is from about 0.75% to about 2.0%.

23. The method of claim 15, wherein the concentration of the corticosteroid in the composition is about 1.5 % hydrocortisone, 0.05% triamcinolone, 0.075% betamethasone dipropionate, or 0.04% clobetasol.

24. The method of claim 15, wherein the concentration of the sulfur in the composition is from about 0.25% to about 2.0%.

25. The method of claim 24, wherein the concentration of the sulfur in the composition is about 0.75%.

26. The method of claim 15, which further comprises instructional material for the use of said method.

27. The method of claim 15, wherein the condition is selected from the group consisting of itching, perioral dermatitis, psorasis, eczema, pruritis, seborrheic dermatitis, acne, rashes, skin irritation, skin scaling and sun damage.

28. The method of claim 15, wherein the user is able to vary the relative amount of each composition to be applied to the skin.

29. A method for the treatment of a skin condition in a user in need of such treatment, said method comprising:
a. providing the system of claim 1;
b. determining the relative amounts of each composition to be applied to the skin; and
c. applying the determined relative amounts of each composition to the skin.

30. The method of claim 29, wherein each composition is contained in separate containers associated with each other, and wherein each of said containers comprises means for dispensing the composition contained therein.

31. The method of claim 29, wherein each composition is contained in a separate pump bottle, and said pump bottles are packaged together in a box or tray.

32. The method of claim 29, wherein the compositions are contained in separate compartments within a vessel.

33. The method of claim 29, wherein the corticosteroid comprises hydrocortisone.

34. The method of claim 29, wherein the corticosteroid comprises hydrocortisone acetate, triamcinolone, betamethasone dipropionate, or clobetasol.

35. The method of claim 29, wherein the sulfur comprises precipitated sulfur.

36. The method of claim 29, wherein the concentration of the corticosteroid in the composition is from about 0.75% to about 2.0%.

37. The method of claim 36, wherein the concentration of the corticosteroid in the composition is about 1.5%.

38. The method of claim 29, wherein the concentration of the sulfur in the composition is from about 0.25% to about 2.0%.

39. The method of claim 38, wherein the concentration of the sulfur in the composition is about 0.75%.

40. The method of claim 29, which further comprises instructional material for the use of said method.

41. The method of claim 29, wherein the condition is selected from the group consisting of itching, perioral dermatitis, eczema, psorasis, pruritis, seborrheic dermatitis, acne, rashes, skin irritation, skin scaling and sun damage.

42. The method of claim 29, wherein the user is able to vary the relative amount of each composition to be applied to the skin.

43. A method for determining a suitable composition for application to the skin of a user in need of such application, said method comprising:
a. providing the system of claim 1;
b. forming a first composition by mixing an amount of the corticosteroid composition and an amount of the sulfur composition, wherein the first composition formed has a higher proportion of corticosteroid and a lower proportion of sulfur as compared to subsequently formed compositions;
c. applying said first composition to the skin of said user;
d. determining the user's tolerance and response for said first composition;
e. if the first composition is tolerated by the user, forming a second composition by mixing an amount of the corticosteroid composition and an amount of the sulfur composition, wherein the second composition has a lower proportion of corticosteroid and a higher proportion of sulfur as compared to the first composition.

44. The method of claim 43, which further comprises:
f. determining the user's tolerance for said second composition; and
g. if the second composition is tolerated by the user, forming a third composition by mixing an amount of the corticosteroid composition and an amount of the sulfur composition, wherein the third composition has a lower proportion of corticosteroid and a higher proportion of sulfur as compared to the second composition.

45. The method of claim 43, wherein at least one of said compositions is formed in the user's hand.

46. The method of claim 43, wherein the first composition is applied to the user's skin at least once daily.

47. The method of claim 46, wherein the first composition is applied to the user's skin for a period of at least about two weeks.

48. The method of claim 47, wherein the first composition is applied to the user's skin for a period of from about two weeks to about four weeks.

49. The method of claim 43, wherein the second composition is applied to the user's skin at least once daily.

50. The method of claim 49, wherein the second composition is applied to the user's skin for a period of at least about two weeks.

51. The method of claim 50, wherein the second composition is applied to the user's skin for a period of from about two weeks to about four weeks.

52. The method of claim 43, wherein the system is contained in separate containers associated with each other, and wherein each of said containers comprises means for dispensing the contents thereof.

53. The method of claim 43, wherein each of the corticosteroid and the sulfur within the system is contained in a separate pump bottle, and said pump bottles are packaged together in a box or tray.

54. The method of claim 43, wherein each of the corticosteroid and the sulfur within the system is contained in separate compartments within a vessel.

55. The method of claim 43, wherein the corticosteroid comprises hydrocortisone, triamcinolone, betamethasone dipropionate, or clobetasol.

56. The method of claim 43, wherein the corticosteroid comprises hydrocortisone acetate.

57. The method of claim 43, wherein the sulfur comprises precipitated sulfur.

58. The method of claim 43, wherein the concentration of the corticosteroid in the composition is from about 0.75% to about 2.0%.

59. The method of claim 58, wherein the concentration of the corticosteroid in the composition is about 1.5%.

60. The method of claim 43, wherein the concentration of the sulfur in the composition is from about 0.25% to about 2.0%.

61. The method of claim 60, wherein the concentration of the sulfur in the composition is about 0.75%.

62. The method of claim 43, wherein the system further comprises instructional material for the use of said method.

63. The method of claim 43, wherein the user has a condition selected from the group consisting of itching, perioral dermatitis, eczema, psorasis, pruritis, seborrheic dermatitis, acne, rashes, skin irritation, skin scaling and sun damage.
